(19) United States
(12) Patent Application Publication
(10) Pub. No.: US 2007/0061016 A1
(43) Pub. Date: Mar. 15, 2007

(54) Foot prosthetic and methods of use

(75) Inventors: Arthur Kuo, Ann Arbor, MI (US); Steven Collins, Ann Arbor, MI (US)

Correspondence Address: Medlen & Carroll. LLP;Suite 350, 101 Howard Street, San Francisco, CA, 94105, US

(73) Assignee: Regents of the University of Michigan, Ann Arbor, MI (US)

(21) Appl. No.: 11/498,669

(22) Filed: Aug. 3, 2006

Related U.S. Application Data
(60) Provisional application No. 60/705,019, filed on Aug. 3, 2005.

(51) Int. Cl. A61F 2/66; A61F 2/70

(52) U.S. Cl. 623/24; 623/55

(57) ABSTRACT

The present invention relates generally to prosthetic devices. In particular, the present invention describes intelligent (e.g., microprocessor controlled) foot prostheses configured to actively store and release energy associated with walking. The foot prostheses of the present invention reduce the energy required during ambulation for amputees requiring foot prostheses.

The present application claims priority to U.S. Provisional Patent Application Ser. No. 60/705,019 filed Aug. 3, 2005, which is herein incorporated by reference in its entirety.

### FIELD OF THE INVENTION

The present invention relates generally to prosthetic devices. In particular, the present invention describes intelligent (e.g., microprocessor controlled) foot prostheses configured to actively store and release energy associated with walking. The foot prostheses of the present invention reduce the energy required during ambulation for amputees requiring foot prostheses.

### BACKGROUND OF THE INVENTION

Over one million persons in the U.S. live with the absence of a limb (National Center for Health Statistics, 1993). Many of these are lower limb amputees, and an estimated 173,000 use an artificial foot or leg (National Center for Health Statistics, 1994). Below-knee amputees make up the majority of this group, and together with above-knee amputees comprise over 80% of amputees. Above-knee amputees use prosthetic knees, which use a range of technologies ranging from passive hydraulic and pneumatic devices, to microprocessor controlled systems that can actively brake the knee. Both above- and below-knee amputees use prosthetic feet, which are generally based on simpler technologies that do not include microprocessor control. All amputees expend more energy than able-bodied persons when walking at the same speed, 20-30% more for unilateral below-knee amputees and still more for above-knee bilateral populations. Young healthy traumatic amputees can tolerate this increase reasonably well, but most amputations are for vascular reasons (e.g., from complications associated with diabetes), and many of these patients have cardiocirculatory problems that limit their energy producing capacity. Vascular amputees experience substantially limited mobility, and would benefit significantly from advanced prostheses if their walking efficiency could be improved. What is needed are improved foot prostheses designed to improve walking and running for amputees.

### SUMMARY OF THE INVENTION

The present invention relates generally to prosthetic devices. In particular, the present invention describes intelligent (e.g., microprocessor controlled) foot prostheses configured to actively store and release energy associated with walking. The foot prostheses of the present invention reduce the energy required during ambulation for amputees requiring foot prostheses. The present invention provides systems, methods, and kits comprising intelligent foot prosthetic devices, employing controlled energy storage and release technologies. Such technology allow for improving the energy efficiency of prosthetic feet by incorporating mechanistic control to adjust the timing of energy release from an elastic mechanism. Unlike currently available prosthetic feet, the controlled energy storage and release technology allows walking, for example, with greater energy efficiency and comfort.

In certain embodiments, the present invention provides a prosthetic foot device, wherein the prosthetic foot device comprises a distal portion engaging a proximal portion at a central pivot point, wherein the distal portion has therein a latch spring positioned between a top portion and a bottom portion, wherein the latch spring is designed to assume a locked latch spring formation and a released latch spring formation, wherein bearing of weight onto the distal portion causes the latch spring to assume a locked latch spring formation, wherein releasing of weight from the distal portion causes the latch spring to assume a released latch spring formation.

In some embodiments, the prosthetic foot device is configured for attachment onto a leg (e.g., an amputated leg). In some embodiments, the bearing of weight onto the distal portion corresponds to a stepping down movement. In some embodiments, the releasing of weight from the distal portion corresponds to a pushing off movement.

In some embodiments, the device further comprises a microprocessor (e.g., micro-electrical mechanical system), wherein the formation of the latch spring is controlled by the microprocessor. In some embodiments, the microprocessor is battery powered. In some embodiments, the microprocessor comprises a distal portion sensor configured to alert the microprocessor of a weight bearing status.

In some embodiments, the assumption of a released latch spring position pushes the proximal portion in a plantarflexion direction. In some embodiments, the latch spring is constructed of a carbon fiber and resin composite. In some embodiments, the prosthetic device is designed for placement within a shoe.

In certain embodiments, the present invention provides a foot prosthesis having therein a microprocessor controlling a latch spring, wherein the microprocessor regulates the amount of compression the latch spring undergoes upon bearing of weight, and wherein the microprocessor regulates the amount of release the latch spring undergoes upon a reduction in amount of weight bore by the latch spring. In some embodiments, the microprocessor controls the timing of when the latch spring compresses or decompresses.

In certain embodiments, the present invention provides kits and systems comprising the foot prostheses of the present invention. In certain embodiments, the present invention provides methods (e.g., medical and research based) utilizing the foot prostheses of the present invention.

In certain embodiments, the present invention provides a prosthetic device comprising a toe plate and a heel plate, the toe plate and heel plate pivotably attached to one-another; a spring disposed between the toe plate and the heel plate, wherein exertion of force on the toe or heel plate compresses the spring; and at least one latch attached to the toe plate or the heel plate such that when the spring is compressed, the at least one latch engages the toe plate and/or the heel plate to maintain compression of the spring thereby storing energy that can be released upon disengagement of the latch. In some embodiments, the prosthetic device further comprises a microprocessor, the microprocessor configured to control the disengagement of the latch. In some embodiments, the prosthetic device is configured for attachment onto a leg (e.g., a below the knee amputated leg).

In some embodiments, the exertion of force onto the toe plate or the heel plate corresponds to a stepping down movement. In some embodiments, the microprocessor controlled latch disengagement is timed to match a lifting off motion during walking. In some embodiments, the microprocessor is a micro-electrical mechanical system. In some embodiments, the microprocessor is battery powered. In some embodiments, the microprocessor controlled latch disengagement pushes the toe plate in a plantarflexion direction.

In some embodiments, the toe plate and heel plate is constructed of a carbon fiber and resin composite. In some embodiments, the prosthetic device is designed for placement within a shoe. In some embodiments, the microprocessor controlled latch disengagement permits the release of energy collected at the heel plate upon the toe plate.

In certain embodiments, the present invention provides a method of facilitating walking with a prosthetic foot comprising providing a prosthetic foot comprising a toe plate and a heel plate, a spring disposed between the toe plate and the heel plate, the compression and release of the spring controlled by a microprocessor; allowing a force to be exerted on the heel plate such that the spring is compressed; and via the microprocessor, releasing the spring such that the energy captured upon compression of the spring is released via the toe plate.

### BRIEF DESCRIPTION OF THE DRAWINGS

FIGS. 1a and 1b depict a foot prosthesis embodiment of the present invention.

FIG. 2 shows side perspectives of additional foot prosthesis embodiments of the present invention.

FIG. 3 shows actions of an intelligent prosthetic foot during the stance phase. The prosthetic has separate heel and forefoot surfaces, both hinged about a pivot located at mid-foot. The forefoot surface extends beyond the pivot and can be locked at the top end of the spring. (a.) At heel strike, the heel section comes into contact with ground, so that during the (b.) load acceptance phase, the spring compresses and stores energy. A latch spring locks the heel at the end of load acceptance, and the foot continues rotating forward during (c.) mid-stance. But during the (d.) push-off phase, the forefoot is released and the spring energy pushes the forefoot in the plantarflexion direction, culminating in (e.) toe-off.

FIG. 4 shows a detailed diagram of prosthesis simulator with controlled energy release. The device (at left) consists of a leaf spring that pivots beneath the foot plate, which supports the foot and ankle immobilizer. The leaf spring flexes when the load of the body acts on the heel or toe. A latch spring mechanism at either end captures this flexure with a ratchet action, and a microprocessor controls the release of the stored energy. The latch mechanism (at right) is a friction ratchet that rectifies motion of the load bar with a guide slot. A solenoid trigger can release the load bar by changing the angle of the guide slot.

FIG. 5 (left) shows net metabolic power consumed while walking with different foot prostheses: Controlled Energy Storage and Return (CESR) prototype and conventional Solid Ankle Cushion Heel (SACH) foot. FIG. 5 (rights) shows push-off work performed on body center of mass. The CESR significantly reduced metabolic rate and increased push-off work compared to The SACH foot.

FIG. 6 shows the average vertical ground reaction forces over one stride. Prototype CESR prosthesis yielded more normal forces (greater push-off and lower collision) than the SACH foot, for the ipsi- to contralateral transition (about 50-60% stride).

FIG. 7 depicts a foot prosthesis embodiment of the present invention.

### DETAILED DESCRIPTION

The present invention provides foot prostheses designed to reduce the energy consumption of walking for amputees. Prostheses and technology related to prostheses have contemplated and described numerous designs with the goal of obtaining a device capable of assisting an amputee in energy efficient ambulation (see, e.g., Kuo, A. D. (2005) Science, 309(5741): 1686-1687; Kuo, A. D. (2005) Journal of Neural Engineering, 2: S235-S249; Kuo, A. D., et al, (2005) Exercise and Sport Sciences Reviews, 33: 88-97; Doke, J., Donelan, J. M., and Kuo, A. D. (2005) Journal of Experimental Biology, 208: 439-445; Donelan, J. M., et al., (2004) Journal of Biomechanics, 37: 827-835; Park, S., Horak, F. B., and Kuo, A. D. (2004) Experimental Brain Research, 154: 417-427; Gard, S. A., Miff, S. C., and Kuo, A. D. (2004) Human Movement Science, 22: 597-610; Dean, J. D., Alexander, N. B., and Kuo, A. D. (2004) Journal of Gerontology: Medical Sciences, 59A: 286-292; Donelan, J. M., Kram, R., and Kuo, A. D. (2002) Journal of Experimental Biology, 205: 3717-3727; Kuo, A. D. (2002) Motor Control, 6: 129-145; Donelan, J. M., Kram, R., and Kuo, A. D. (2002) Journal of Biomechanics, 35: 117-124; Kuo, A. D. (2002) Journal of Biomechanical Engineering, 124: 113-120; Speers, R. A., Kuo, A. D. (2002) Gait and Posture, 16: 20-30; Donelan, J. M., Kram, R., and Kuo, A. D. (2001) Proceedings of the Royal Society of London, Series B, 268: 1985-1992; Kuo, A. D. (2001) Journal of Biomechanical Engineering, 123: 264-269; Bauby, C. E., and Kuo, A. D. (2000) Journal of Biomechanics, 33: 1433-1440; Kuo, A. D. (1999) International Journal of Robotics Research, 18(9): 917-930; Speers, R. A., Shepard, N. T., Kuo, A. D. (1999) J. Vestibular Research, 9 (6): 435-444; Kuo, A. D., Speers, R. A., Peterka, R. J., and Horak, F. B. (1998) Experimental Brain Research, 122: 185-195; Kuo, A. D. (1998) J. Biomechanical Engineering, 120(1): 148-159; Kuo, A. D. (1995) IEEE Transactions on Biomedical Engineering, 42: 87-101; Adams, J. M. and Perry, J. (1992) Prosthetics. In: (Perry, J., ed.) Gait Analysis: Normal and Pathological Function. SLACK Inc.: Thorofare, N.J. pp. 165-200; Barr, A. E., Siegel, K. L., Danoff, J. V., McGarvey, C. L. 3rd, Tomasko, A., Sable, I., Stanhope, S. J. (1992) Biomechanical comparison of the energy-storing capabilities of SACH and Carbon Copy II prosthetic feet during the stance phase of gait in a person with below-knee amputation” Physical Therapy 72:344-54; Buckley, J. G., et al., (2002) Arch. Phys. Med. Rehabil. 83: 576-580; Buckley, J. G., Spence, W. D., Solomonidis, S. E. (1997) Arch. Phys. Med. Rehabil. 78: 330-333; Casillas, J. M. Dulieu (1995) Arch. Phys. Rehabil. 76: 39-44; Colborne, G. R., et al., (1992) Am. J. Phys. Med. Rehabil. 92: 272-278; Collins, S. H., Wisse, M., Ruina, A. (2001) Int. J. Robot. Res. 20: 607-615; Donelan, J. M., Kram, R., and Kuo, A. D. (2002a) Mechanical work for step-to-step transitions is a major determinant of the metabolic cost of human walking. Journal of Experimental Biology, 205: 3717-3727; Donelan, J. M., Kram, R., and Kuo, A. D. (2002b) Journal of Biomechanics, 35: 117-1241; Donelan, J. M., Kram, R., and Kuo, A. D. (2001) Proc. Royal Soc. Lond. B, 268: 1985-1992; Farley, C. T., Gonzalez, O. (1996) J Biomech. 29:181-186; Fukunaga, T., Kubo, K., Kawakami, Y., Fukashiro, S., Kanehisa, H., Maganaris, C. N. (2001) Proc. R. Soc. Lond. B 268: 229-233; Gailey, R. S., Wenger, M. A., Raya, M., Kirk, N., Erbs, K., Spryopoulos, P., and Nash, M. S. (1994) Prosthet. Orthot. Int. 18: 84-91; Gailey, R. S., Nash, M. S., Atchley, T. A., Zilmer, R. M., Moline-Little, G. R., Morris-Cresswell, N., Siebert, L. I. (1997) Prosthet. Orthot. Intl. 21: 9-16; Geil, M. D., Parnianpour, M., Quesada, P., Berme, N., Simon, S. (2000) Journal of Biomechanics 33: 1745-50; Herbert, L. M., Engsberg, J. R., Tedford, K. G., Grimston, S. K. (1994) Physical Therapy 74: 943; Herr, H. and N. Langman. (1997) Journal of the International Society for Structural and Multidisciplinary Optimization (ISSMO). 13: 65-67; Huang, G. F., Choum Y, L., Su, F. C. (2000) Gait & Posture 12: 162-8; James, U. (1973) Scand. J. Rehabil. Med. 5: 71-80; Kuo, A. D. (2002) Journal of Biomechanical Engineering, 124: 113-120; Kuo, A. D. (2001) Journal of Biomechanical Engineering, 123: 264-269; Lee, C. R., Farley, C. T. (1998) J. Exp. Biol. 201:2935-2944; Lehmann, J. F., Price, R., Boswell-Bessette, S., Dralle, A., Questad, K., deLateur, B. J. (1993) Arch. Phys. Med. Rehabil. 74: 1225-1231; Lehmann, J. F., Price, R., Boswell-Bessette, S., Dralle, A., Questad, K. (1993) Arch. Phys. Med. Rehabil. 74: 853-861; Lemaire, E. D., Nielen, D., and Paquin, M. A. (2000) Arch. Phys. Med. Rehabil. 81: 840-843; Molen, N. H. (1973) Int. Z. Angew. Physiol. 31: 173; Postema, K., Hermens, H. J. de Vries, J., Koopman, H. F., Eisma, W. H. (1997) Prosthetics and Orthotics International 21: 17-27; Powers, C. M., Boyd, L. A., Fontaine, C., Perry, J. (1996) Phys. Ther. 76: 369-377; Prince, F., Winter, D. A., Sjonnensen, G., Powell, C., Wheeldon, R. K. (1998) Journal of Rehabilitation Research and Development 35:177-85; Romo, H. D. (2000) Physical Medicine and Rehabilitation Clinics of North America 11: 595-607; Roberts, T. J, Kram, R., Weyand, P. G., Taylor, C. R. (1998) J Exp Biol. 201:2745-2751; Rossi, D. A., Doyle, W., and Skinner, H. B. (1995) Journal of Rehabilitation Research 32: 120-127; Scherer, R. F., Dowling, J. J., Robinson, M., Frost, G. F., McLean, K. (1999) Journal of Prosthetics and Orthotics 11: 38-42; Seymour, R., Ordway, N., Bachand, A., Rufa, A., Wetherby, D. (2002) A Comparison of the 3C100 C-leg prosthetic knee joint to conventional hydraulic prosthetic knees: A kinematic, kinetic, physiological, and functional outcome survey pilot study. In: Gait and Clinical Movement Analysis Society, 7^{th} Annual Meeting, Chattanooga, Tenn.; Thomas, S. S., Buckon, C. E., Helper, D., Turner, N., Moor, M., Krajbich, J. I. (2000) Journal of Prosthetics and Orthotics 12:9-14; Torburn, L., Powers, C. M., Guiterrez, R., Perry, J. (1995) Journal of Rehabilitation Research and Development 32:111-9; Waters, R. L. and Mulroy, S. (1999) Gait and Posture 9: 207-231; Whittle, M. W. (1996) Gait Analysis: An Introduction, 2^{nd} ed. Oxford: Butterworth-Heinemann; and U.S. Pat. Nos. 4,547,913, 5,037,444, 5,258,038, 6,029,374, 6,602,295, and 6,007,582; each of which is herein incorporated by reference in their entireties).

The following description describes prosthetic devices of the present invention in terms of foot prostheses. It should be noted, however, that the concepts and devices of the present invention are not limited to foot prostheses. Indeed, the present invention contemplates, for example, intelligent prostheses for elbow, ankle, knee, hip, wrist, shoulder and neck. In addition, the following description is in terms of amputee subjects. The concepts and devices of the present invention, however, could be applied to any disorder or situation requiring assistance in, for example, ambulation (e.g., stroke patients, paralysis patients, debilitated patients, rehabilitation patients).

The present invention is not limited to a particular foot prosthesis design or configuration. In some embodiments, the present invention provides foot prostheses with an “intelligent” (e.g., microprocessor controlled) design configured to reduce energy consumption typically required for an amputee while walking. The foot prostheses of the present invention provide significant improvements over currently available foot prostheses. In particular, the foot prostheses of the present invention employ an intelligent design (e.g., an actively controlled energy and storage release design) so as to store and release energy through use of, for example, a latch spring mechanism controlled by a microprocessor. As such, the foot prostheses of the present invention employ an “active” design (e.g., not passive) to provide articulation, cushioning against heel impact, and elastic energy return.

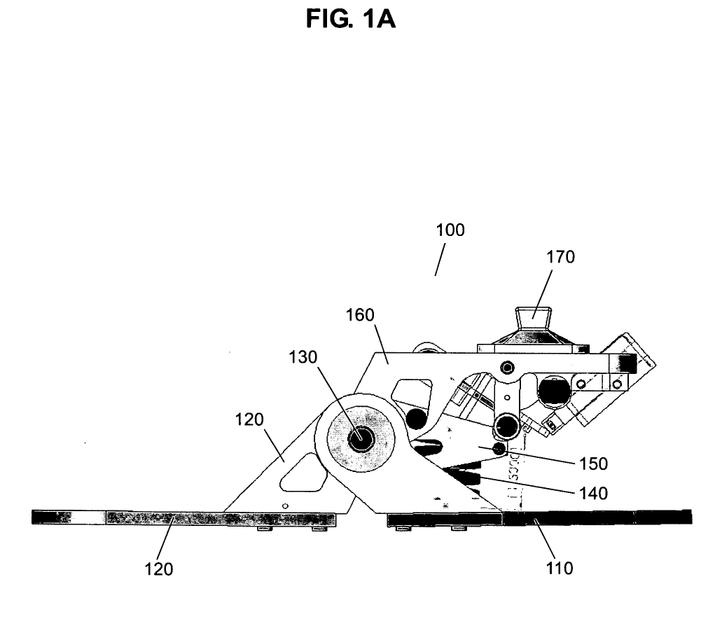

FIG. 1 shows a side perspective of a foot prosthesis of the present invention. The foot prosthesis 100 is not limited to a particular size. In some embodiments, the size of the foot prosthesis 100 is variable (e.g., so as to match a user's non-amputated foot). In some embodiments, the foot prosthesis 100 is sized so as to bear a user's weight during running or walking. The foot prosthesis 100 is not limited to a particular composition (e.g., plastic, Kevlar, titanium, etc.). The foot prosthesis 100 has a heel plate 110 and a toe plate 120. In preferred embodiments, the foot prosthesis 100 is designed such that as weight is provided onto the heel plate 110 (e.g., during walking), energy is stored, and as that weight is lifted off of the heel plate 110 (e.g., during walking), that energy is released so as to reduce the amount of energy required during walking (described in more detail below).

Still referring to FIG. 1, the heel plate 110 is pivotably attached to the toe plate 120, preferably via a central axis 130. The foot prosthesis 100 further comprises a spring 140 disposed between a spring extension 150 of the toe plate 120 and the heel plate 110. The foot prosthesis 100 also includes an adapter plate 160 that is also pivotably attached to the central axis 130. The adapter plate 160 comprises a lug 170 for connecting a prosthetic leg (not shown) to the foot prosthesis 100. The foot prosthesis 100 further comprises a toe latch and heel latch that are engaged when the spring 140 is compressed and released during walking so that energy stored from compression of the spring 140 during heel strike is transferred to and released from the toe plate. In some embodiments, the latches are configured to lock upon full compression of the spring. In some embodiments, the heel plate 110 has therein weight detection sensor(s). The present invention is not limited to a particular type, kind, or size of sensor. In some embodiments, the sensors are able to communicate (e.g., wirelessly or via wires) with a microprocessor for purposes of controlling the timed release of the latches so that energy stored from the heel strike is released via the toe plate 120. The present invention is not limited to a particular type, kind or size of a microprocessor. In some embodiments, the microprocessor is able to auto-sense its state using a small number of sensors. In some embodiments, the microprocessor is programmed to control when the spring 140 compresses, to what degree the spring 140 compresses, for how long the spring 140 remains compressed, when the spring 140 is locked, for how long the spring 140 is locked, at what point the locked spring 140 is released to an unlocked state, and the ease of which the spring 140 is able to compress and decompress. In some embodiments, the microprocessor controls the spring 140 so as to provide active energy storage and release of the foot prosthesis 100.

For example, in some embodiments, the foot prosthesis 100 is designed such that as is exerted on the adapter plate 160, sensors are able to detect the assumption of force onto the heel plate 110, provide that information to the microprocessor, and the microprocessor 180 is able to lock the spring at a certain compression. As the force is removed from the heel plate 110, the sensors detect the weight change and provide that information to the microprocessor, wherein the microprocessor releases the locked, compressed spring 140 thereby providing that energy to assist in a walking or running gait. In some embodiments, the microprocessor can be configured to lock and release the spring 140 at variable weight assumption thresholds (e.g., upon assumption of 1 pound, 10 pounds, 15 pounds, 20 pounds, etc; or upon release of 1 pound, 10 pounds, 15 pounds, 20 pounds, etc). In some embodiments, the microprocessor can be configured to not lock the spring 140 so as to achieve a passive configuration. In some embodiments, the microprocessor is configured to release a locked (e.g., compressed) latch spring 140 at the apex of lift-off so as to provide maximum energy to the user during ambulation. In preferred embodiments, the energy storage and release aspects of the foot prosthesis 100 allows a user to conserve more energy and walk/run easier than with using currently available foot prostheses.

The present invention is not limited to the foot prosthetic embodiment described in FIG. 1. In some embodiments, the foot prostheses include additional sensors (e.g., accelerometers, gyroscopes) to detect and compensate for changes in ground slope. In some embodiments, it is contemplated that a user may have both feet amputated, and require two foot prosthesis. In such situations, the foot prostheses are configured to communicate with each other (e.g., via Bluetooth) for purposes of coordinating walking and/or running motion (e.g., to coordinate energy capture and release). In some embodiments, the foot prostheses have therein separate motors for providing its own movement (e.g., in situations wherein a person may be paralyzed). In some embodiments, the foot prostheses are configured to attach, by any method, style or technique, to any portion of a subject's leg (e.g., below the knee, below the shin, above the knee, etc.) so as to secure the foot prosthesis onto a user. The foot prostheses of the present invention are not limited to a number or type of accessories.

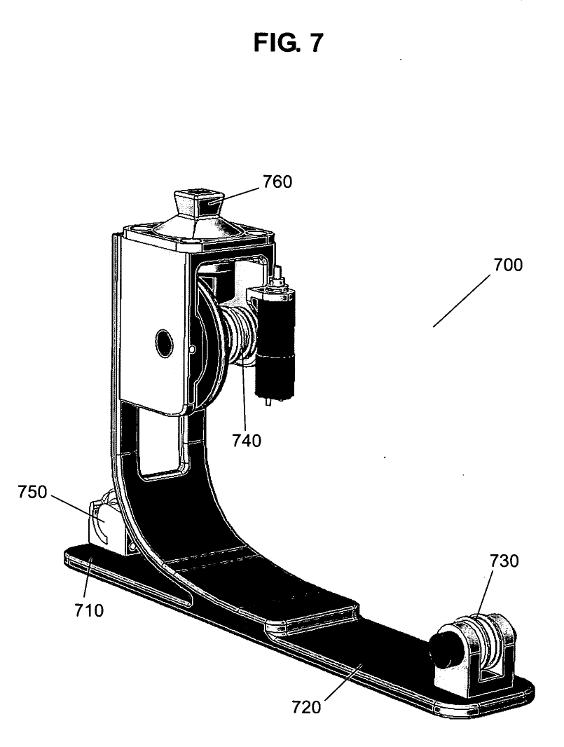

FIG. 7 depicts an additional embodiment. In this embodiment, the foot prosthesis 700. The foot prosthesis 700 is preferably formed from carbon fiber and comprises a heel portion 710 and toe portion 720. In preferred embodiments, the heel and toe portions 710 and 720 form compressable leaf springs. The foot prosthesis 700 further comprises a series of pulleys 730, 740, and 750. The pulleys 730, 740 and 750 are preferably connected by a cable (not shown). The foot prosthesis 700 further comprises a lug 760 for attaching to a leg or a prosthetic leg. When a force is exerted on the foot prosthesis 700, such a downward force exerted on the lug 760, the heel portion 710 captures the energy is maintained in a compressed state via action of the cable and pulleys 730, 740 and 750 which can be locked to maintain a compressed state. The pulleys can preferably be released so that the energy stored in the heel portion 710 is released via the toe portion 720. In preferred embodiments, the locking and release of the pulleys 730, 740 and 750 is controlled by a microprocessor essentially as described in detail above.

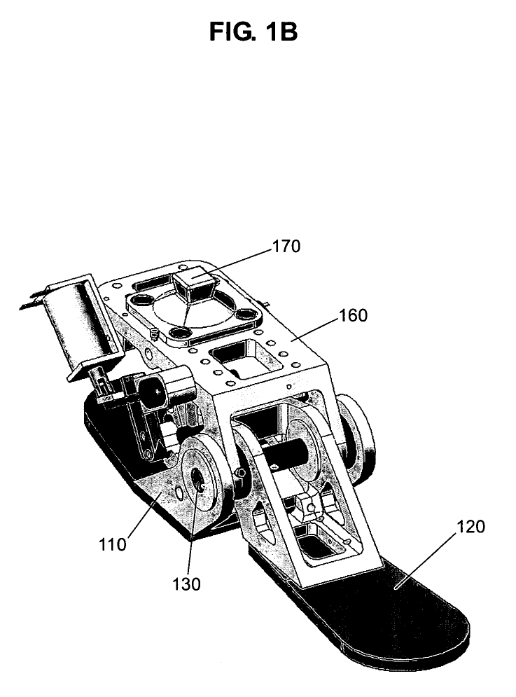

FIG. 2A shows a foot prosthesis utilizing cables to engage the latch spring. In such embodiments, the cables are kept in tension by an internal take-up reel and two latches that can release either end of the latch spring. FIG. 2B shows a foot prosthesis of the present invention having a dual pivot design, in which energy is stored in a linear compression spring. The heel and forefoot plates are hinged by a dual pivot at mid-foot, and each plate is latched separately (internal to mechanism), so that each plate can be locked or released independently. At heel strike, the heel plate moves and then is latched to capture the spring compression, while the forefoot plate is locked. After stance, the heel plate is kept locked, but the forefoot plate is released, and the spring pushes it into tarflexion. FIG. 2C shows a latched axial spring design, for use with an existing prosthetic foot. Computer control, for example, of spring release allows the energy return to be timed appropriate to walking speed.

Persons who have lost a lower limb have restricted mobility, and expend 20-30% more energy to walk at the same speed as able-bodied individuals. Currently available foot prostheses (e.g., SACH foot prostheses, DER foot prostheses) employ passive mechanisms to provide articulation, cushioning against heel impact, and elastic energy return. Such prostheses are not as technologically sophisticated as, for example, intelligent knees, which improve gait by actively controlling braking of the knee, resulting in a 5-10% decrease in energy cost for walking. Currently available foot prostheses (e.g., energy storing feet) have not shown consistent energy improvements. Currently available foot prostheses, for example, have a static stiffness yet must simultaneously satisfy numerous objectives that require different stiffnesses at different walking speeds, and very high stiffness for standing. A more efficient gait is therefore difficult to achieve with a passive prosthesis.

In some embodiments, the foot prostheses of the present invention are designed to significantly improve the efficiency of an amputee gait. Such foot prostheses are designed to, for example, store elastic energy after a foot strikes the ground through capturing of the energy via a latch spring mechanism, and, releasing it later in the gait cycle, coinciding with the push-off phase of able-bodied walking. Experiments conducted during the course of the present invention indicate that the proper timing of energy release in one foot yields significant savings in energy, and reduces the impact of the other foot with the ground, thereby improving comfort.

Currently available foot prostheses are technically simple, and rely on purely passive mechanical components. A widely used foot is the Solid Ankle Cushioned Heel (SACH) foot. The SACH foot is mostly solid except for a compressible heel wedge, which dissipates energy during the load acceptance phase directly after heel strike. In able-bodied individuals, the center of mass is moving forward and down during this phase, with energy absorbed by the stance ankle and knee. The SACH heel lessens the impact of heel strike, followed by a smooth transition to mid-stance, with reduced vibrations transmitted to the stump. Foot prostheses utilizing Dynamic Elastic Response (DER) technology store and return energy using a carbon fiber leaf spring for the foot, or with elastic bumpers acting on hinged heel and forefoot surfaces. There exist other foot prostheses that provide limited articulation, but these are also purely passive systems. The simplest articulation is in a single-axis foot (e.g., Kingsley), pre-dating the SACH foot and providing limited plantar-/dorsi-flexion of the ankle, with elastic bumpers controlling and limiting that motion. Plantarflexion following heel strike allows the center of pressure under the foot to progress forward more quickly, which helps to extend the knee.

In some embodiments, the foot prostheses of the present invention have a flexible composition, thereby providing an additional improvement over currently available foot prostheses. Walking differs from running in several ways. First, the center of mass is at its highest point at mid-stance, implying any energy stored heel strike must immediately be returned. This immediate return indicates that no energy remains to assist in push-off, when a large amount of positive work is performed by the able-bodied person's trailing leg (see Whittle, 1996). Second, the ground contact time during walking is considerably longer than during running. This implies that the stiffness and natural frequency of oscillation appropriate for running are too high for walking. A lower stiffness would require a much larger amount of travel, which is unacceptable if the gait is to resemble normal human walking with the center of mass at its highest point at mid-stance. Indeed, current energy-storing foot prosthetics may not return energy at the proper time, due to an overly high natural frequency of oscillation. Conventional energy-storing feet are also constrained by the need for relatively high stiffness to provide a stable platform for standing. A constant stiffness is therefore unlikely to simultaneously satisfy the requirements for walking at a variety of speeds, running, and stable standing.

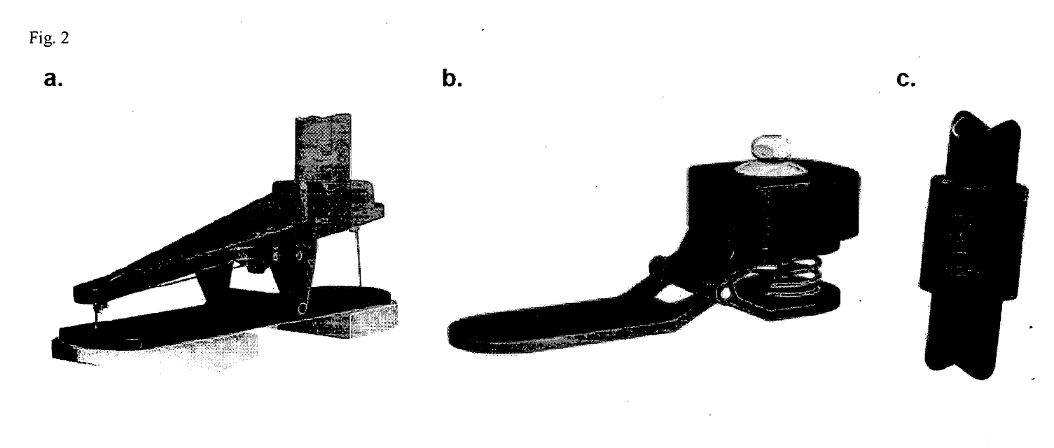

The characteristics of walking present an opportunity for energy storage and release in an intelligent mechanism. In experiments conducted during the course of the present invention, it was shown that the energy dissipation that occurs in an able-bodied person's load acceptance phase can be stored in the spring of a prosthetic foot, provided the energy is captured momentarily. At the ankle, the energy would be in the form of negative work as the foot falls flat. In some embodiments, the device provides an actuated ratchet for locking a spring storing this energy (described in more detail below). In such embodiments, the energy is retained past stance, and released during push-off (see FIG. 3). This storage and release, rather than attempting to mimic actual human physiology, instead mimics the mechanical actions of negative work during load acceptance, and positive work during push-off. The timed release could also be interpreted as a means to artificially manipulate the natural frequency of oscillation, so that it could be modulated according to walking speed. The locking mechanism also makes it possible for the spring to be locked out, as would be desirable for standing. The technical requirements of such a mechanism are first, an ability to store energy and capture it; and second, to be able to perform negative and then positive work while exerting torque in opposite directions.

Springs normally store and release energy in opposite directions of motion stretching and lengthening, but the same direction of force. An appropriate latch mechanism must store and release energy in the same direction of motion but opposite directions of force, as in producing dorsiflexion torque during the load acceptance (energy storage) phase, and then flexion torque during the push-off (energy release) phase. This spring reversal can be accomplished with two latches, one to release each end of the spring, in concert with an additional light return spring that resets the mechanism between steps. The combined power requirements for capturing, releasing, and reversing spring forces could in principle be quite small, compared to the amount of energy being stored in the spring. This makes such a mechanism feasible for battery power.

Recent measurements indicate that a substantial amount of mechanical energy is dissipated during walking in a manner that could potentially be captured by an intelligent prosthesis. Metabolic energy studies further suggest that humans perform mechanical work which could be reduced if a prosthesis released stored energy at an appropriate time. The present invention is not limited to a particular mechanism. Indeed, an understanding of the mechanism is not necessary to practice the present invention. Nonetheless, it is contemplated that such a storage and release improves upon the energy storage in able-bodied walking. In normal walking, there appears to be relatively little energy return from the load acceptance phase, and then a separate and small energy return from the Achilles tendon during the push-off phase. Energy is stored in the Achilles tendon during mid-stance, as the center of mass moves forward over the leg and the calf muscles (gastrocnemius, soleus) produce force at relatively slow shortening speeds as the tendon stretches. This energy is then quickly released during the push-off phase. However, the amount of energy being stored is estimated to be fairly low. Recent findings form the conceptual basis for the intelligent foot prostheses described in the present invention, suggesting that the energy normally dissipated during load acceptance could in principle be stored and captured by a prosthesis, and then the energy normally produced during push-off could be released from storage. Technological improvements in inexpensive microprocessor control, miniature sensors, electrical energy storage, and lightweight materials, all contribute to the probability of success for an intelligent foot prosthesis.

The foot prostheses of the present invention are designed to reduce the amount of energy needed for an amputee to walk. For example, during the first half of the stance phase in able-bodied walking, mechanical energy is absorbed by the leading leg. The amount of energy absorbed is greater than what can be quantified from joint power alone, because there is also energy absorbed by the shoe, heel, and other flexible structures. The total energy can be summarized by the amount of negative work performed on the center of mass by the leading leg during the load acceptance phase, which was found to be about 15 J per step, or a rate of nearly 30 W for walking at a typical speed of 1.25 m/s. In order to walk at a steady speed, negative work must be restored through an equal amount of positive work, which is per-formed by pushing off with the trailing leg. There appears to be a metabolic cost not only for performing the positive work, but for the negative work as well. In other words, even though the leading leg is performing negative mechanical work, there is a positive metabolic cost associated with it. In able-bodied subjects, it is estimated that the overall metabolic cost for this work to be up to 120 W, or as much as two-thirds of the net metabolic cost of walking. The negative work is normally absorbed by the joints, and also dissipated by the heel and other parts of the leg.

The Dynamic Elastic Response (DER) prosthesis is designed to passively (e.g., spontaneously) store and release energy during the walking process. The intelligent control of the foot prostheses of the present invention add the capability of capturing that energy and releasing it at an opportune moment. In able-bodied gait, the 15 J per step is normally absorbed at the joints and dissipated by the shoe, heel pad, and other parts of the leg. An aim of the intelligent foot prostheses of the present invention is to direct that energy to the latch spring (e.g., approximately 30% of the total negative work can be stored; the spring will store 4-5 J per step). Assuming that friction and the need to reverse the spring force amount to a 50% loss, 2-2.5 J will be returned to the center of mass upon release of the latch spring. This is energy that would otherwise be supplied actively by muscle. At a speed of 1.25 m/s, this amounts to a conservative estimate of 4 W of mechanical power savings. The foot prostheses of the present invention are designed to release this energy during push-off so as to reduce the amount of mechanical work the person must provide, and therefore reduce the metabolic energy expended. For example, the foot prostheses of the present invention will save approximately 16 W of metabolic energy, or about 10% of the net metabolic cost of walking.

### EXAMPLES

### Example I

This example describes the use of a foot prosthetic simulator designed to demonstrate the conceptual advantage of a foot prosthesis utilizing an intelligent design. The simulator was worn on the lower extremity of an able-bodied subject, such that it immobilized the ankle and allowed the attachment of a variety of alternative artificial foot surfaces. It was similar to an ankle foot orthosis, except that it allowed able-bodied persons to simulate prosthetic gait. The primary attachment designed was a spring device which satisfied the mechanical requirements of a controlled-release storing prosthesis. A secondary attachment was designed, to roughly emulate a conventional energy-storing prosthesis. These attachments allowed a single human subject to compare the experience of walking with conventional and controlled-release energy storage, in both unilateral and bilateral configurations. Moreover, these conditions allowed comparison with the same subject's able-bodied gait. The prosthetic simulator device functioned as a test-bed for proving the overall feasibility of the project. A pair of such devices were built, to allow for bilateral testing.

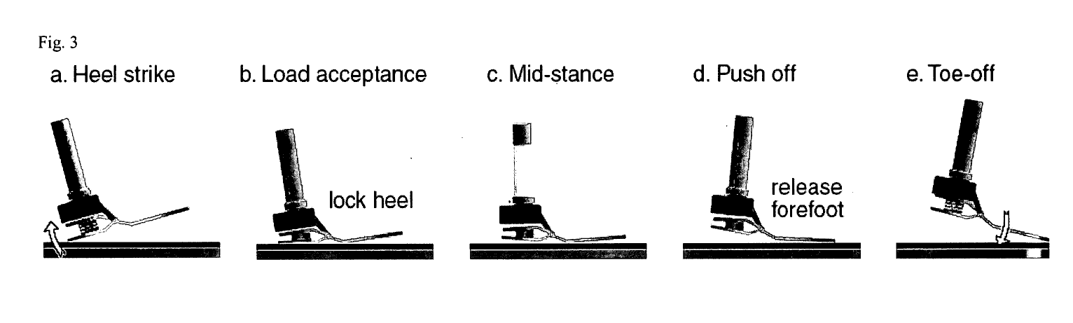

FIG. 4 provides a schematic of the prosthesis simulator. An able-bodied individual's foot and ankle are immobilized on a foot plate with a polyethylene cuff about the shank. Below the foot plate, a carbon fiber leaf spring bends and pivots about the middle of the foot. Two latches, at front and back, can capture and release either end of the spring as commanded by a microprocessor. A layer of capacitive sensors is bonded to the bottom of the leaf spring, providing location of ground contact information. The bottom layer is a thin vibram sole for protection and to prevent slipping.

The main features of the prosthetic simulator are as follows. The ankle is immobilized by a lightweight calf support made of aluminum with a low-density polyethylene cuff, attached to a carbon fiber foot support (foot plate), on which a bicycle racing shoe is mounted. A bicycle racing shoe is specified because it provides an inexpensive foot attachment that is light and stiff, due to a carbon fiber sole, and is also designed to support loads pulling from the sole. The bottom of the platform has attachment points for either the controlled-release energy storing spring, or an unactuated leaf spring that is similar to the foot surface for conventional commercial prostheses.

As shown in FIG. 4, the energy storage mechanism itself has five major parts: the foot plate, a leaf spring, a pivot, and two latch mechanisms. The foot plate is in the form of a U-beam, and provides structural support not only for the foot but also for the leaf spring. The leaf spring is similar to those found in conventional DER prostheses, constructed of a carbon fiber and resin composite with high elasticity and energy return to accompany its light weight and high strength. The pivot, of hardened ground stainless steel, is mounted approximately midway between the heel and toe, approximately 1½ inch below the aluminum platform. The pivot allows the energy that is stored by bending of the spring at the heel, to be released at the toe. The leaf spring is connected to two electronically-controlled mechanisms, each allowing for upward but not downward motion of one end of the spring. The latches capture energy of the spring, as it one end bends under the weight of the body. The mechanism of each latch is similar to that of popular carpentry clamps, capturing the motion of a bar with a guide slot. When the slot is in a normal configuration, the clearance in the slot allows for free motion of the bar in one direction. But reversal of direction pulls the slot into a pivoted position, where it suddenly locks the bar securely. Either end of the leaf spring can be released by a solenoid attached to each latch, actuated by an microcontroller. When both latches are released, a very light return spring pulls the toe end of the leaf spring into a home position, in contact with the aluminum platform. The toe latch can then be set to capture that end of the leaf spring. In this home position, the leaf spring is in position to absorb the load of the body upon heel strike. The entire mechanism adds no more than 2″ in extra height to the subject, and to weigh no more than 2.5 kg.

The prosthesis simulator includes several electronic components. A small 586-based driven microcontroller (TERN, Inc.) provides sensing, timing, and control functions. It receives input from several sensors. These include three inexpensive capacitive load sensors bonded to the bottom surface of the leaf spring, that inform the controller when a foot is under load, and the approximate location (heel, toe, or middle) of that load. Motion sensing are provided by two miniature piezo-based accelerometers and a rate gyroscope, all in dual in-line chip packages. Finally, analog and digital inputs are connected to a handheld remote control, containing a potentiometer and pushbuttons. The user are able to adjust timing of the device's control actions with the potentiometer, and to command the device to perform in different modes of operation with the pushbuttons. A small custom-printed circuit board houses the electronics, with power provided by rechargeable nickel-cadmium batteries and dc voltage regulators. The microcontroller logs data in memory during experimental trials, and then transfers to computers via serial cable.

The energy storage and release action is coordinated with the gait cycle. At the end of the swing phase, the leaf spring is in home position, with the toe latch locked. After heel strike and during the load acceptance phase of gait, the leaf spring is compressed at the heel, and the energy captured by the heel ratchet. Once the energy is stored, the leaf spring is slightly curved, and the subject progresses forward on this surface. After mid-stance, during the push-off phase, the microcontroller releases the toe latch, so that the leaf spring's energy is released after a delay. Moreover, the release occurs at the forward end of the spring, producing a push-off action approximating that of an able-bodied toe. After toe-off, the leaf spring has no load acting on it, and therefore no stored energy, and it is in a final position where the toe is free and the heel is locked. At this point, the microcontroller releases the heel latch and re-engages the toe ratchet. A light return spring brings the mechanism back to its home position, with the toe automatically locked by the ratchet. The device then is in proper configuration for the next heel strike.

This mechanism has several minor design features. One is that the ratchet action of the latch is not a gear-and-pawl type. Rather, the ratchet uses friction, as found in common bar clamps used in carpentry. The friction mechanism locks a translating bar with a hinged slot which is large enough for the bar to pass through easily and with little friction. The bar's motion is rectified (i.e., allowed in only one direction) by the action of the slot when the motion is reversed. Such a mechanism is simple and presents little resistance in the direction of desired motion, yet locks easily and automatically, and can be released with a small force to rotate the slot. This force is provided by the microcontroller-driven solenoid. Another design feature is that a light return spring is needed to bring the leaf spring to home position when both ratchets are released. The return spring will produce negligible force relative to the bending force of the leaf spring, but is sufficient to overcome the slight resistance of the friction ratchet at the toe.

### Example II

This example describes a proposed research protocol utilizing the prosthesis simulator. A simple set of experiments will be used to test the feasibility of controlled-release energy storage. These experiments will be performed on 12 able-bodied young human subjects. Subjects will be recruited by advertisement, with their informed consent and safety ensured. The experiments will test and compare subjects' gait with and without the prosthesis simulator, with and without controlled-release of stored energy. The outcome measures are the metabolic energy expenditure of at a given speed, as well as and ground reaction forces. The subjects will perform multiple walking trials at a given speed of 1 m/s, a slow and comfortable walking speed. These trials will be performed once overground in order to measure ground reaction forces, and then repeated on a treadmill to measure metabolic energy expenditure. The overground trials will also involve measurement of joint motions by a Optotrak motion analysis system. In those trials, subjects will wear a set of infrared markers, using a standard gait analysis standard (e.g., modified Helen Hayes market set). Walking speed will be monitored with a set of trip lights mounted midway through the walkway. Two force plates will record the subjects' foot strikes as they walk past the trip lights. Trials will be repeated if subjects do not maintain the target walking speed within 5%, or if subjects do not step cleanly on the force plates. A minimum of three acceptable trials will be collected at each experimental condition.

The treadmill trials will be performed on a Trackmaster treadmill, set to the same speed as the overground trials. Subjects will walk for six minutes, while their oxygen consumption is recorded with a Vmax metabolic energy analyzing system. Oxygen consumption and carbon dioxide production rates will be recorded for the final three minutes, with the first three minutes used to reach steady state. The combined oxygen and carbon dioxide data will be used to compute the metabolic rate. These trials will be recorded separate from the force plate trials because metabolic energy expenditure requires longer trials than are possible in an overground walkway with force plates, and because it is difficult or impossible to measure the ground reaction forces under the separate legs while subjects walk on a treadmill. Some treadmills do have embedded force plates, but these currently do not provide a full set of forces (three translational forces, three moments) for each leg. It is therefore necessary to perform separate trials, attempting to control for speed and other variables as much as possible.

The data collection will be preceded by a testing phase to allow for setting of control parameters. The controller will release the latches based on timing of gait events. The critical control variable is the timing of the toe release, relative to the timing of forces measured by the capacitive sensors under the leaf spring. An extensive set of informal tests of different phasing schemes will be performed. A candidate timing parameter that can then be tested quantitatively through controlled experiments will be determined.

The experimental conditions are designed to compare multiple variations of each subject's gait. These will include two different able-bodied conditions, two conventional prosthesis conditions, and two controlled-release conditions. The first able-bodied condition will involve subjects walking normally in their own shoes. This will serve as a baseline for all other comparisons. In the second able-bodied condition, subjects will wear a prosthesis simulator on each foot, but without the ankles immobilized, and with the leaf spring locked in the energy-stored position (heel and toe ratchets both locked). This will assist in quantifying the energetic disadvantages of walking while wearing the prosthetic simulators, due to their weight, extra height, and the slightly curved surface of the leaf spring. It is anticipated that energetic costs will be somewhat greater than those for walking in normal shoes. However, modest amounts of added mass do not typically add to the energetic cost of walking.

The conventional prosthesis conditions will make use of a carbon fiber leaf spring, without any controlled release. Although the spring will not be identical to commercial energy-storing designs such as Flex-foot, it will bear an approximate resemblance to the mechanical behaviors of a conventional spring. There will be two conditions without controlled energy release: bilateral and unilateral. In the bilateral case, subjects will wear one prosthesis simulator on each foot. In the unilateral case, subjects will wear a single prosthesis simulator on their dominant foot, and a platform shoe riser on the other foot.

The controlled-release conditions will make use of the full capabilities of the prosthesis simulator, using the release parameters determined from the informal testing phase. Again, the conditions will be bilateral and unilateral. For bilateral trials, subjects will again wear one prosthetic simulator on each foot. In the unilateral trials, subjects will wear prosthetic simulator on one foot and a platform shoe riser on the other foot. In the informal testing phase, it is anticipated that the bilateral and unilateral conditions may favor different toe-release phasing parameters. As such, the two conditions will make use of differing phasing parameters.

### Example III

This example describes an experiment with a prosthesis simulator. Humans actively push off with the trailing leg just before and during the double support phase of walking. Push-off compensates for the energy lost as the leading leg performs negative work during the transition between steps (see, e.g., Donelan, J M, et al. J Exp. Biol. 205: 3717-3727, 2002; herein incorporated by reference in its entirety). Simple models predict that the energy used in walking is strongly linked to the mechanics of this step-to-step transition; pushing off just before double support can theoretically reduce the step-to-step transition work by a factor of four (see, e.g., Kuo, A D. J. Biomech. Eng. 124: 113-120, 2002; herein incorporated by reference in its entirety).

Lower-limb amputees have a reduced capacity for ankle pushoff during walking (see, e.g., Whittle, M W. Gait Analysis: An Introduction, 1996; herein incorporated by reference in its entirety) contributing to a 20-30% greater energy demand than intact individuals (see, e.g., Waters, R L, et al. Gait & Posture. 9(3): 207-231, 1999; herein incorporated by reference in its entirety). A variety of prosthetic feet have been designed with elastic properties to compensate for lost ankle function, but none have significantly reduced the metabolic cost of walking compared to the conventional Solid Ankle Cushion Heel (SACH) foot (see, e.g., Waters, R L, et al. Gait & Posture. 9(3): 207-231, 1999; herein incorporated by reference in its entirety). It was hypothesized that mechanical energy should optimally be stored during load acceptance and released during push-off, as opposed to being spontaneously returned as in existing elastic prostheses. This hypothesis was tested by constructing a prototype prosthetic foot with Controlled Energy Storage and Return (CESR), and by measuring the resulting metabolic cost of walking.

Intact individuals were tested using a foot prosthesis simulator, a boot that securely constrains the ankle and has a foot prosthesis attachment at its base. Each subject wore the prosthesis unilaterally (ipsilateral foot) with a rocker-bottomed lift on the contralateral foot to compensate for the 10 cm height of the prosthesis attachment. 5 male subjects (ages 20-25 yrs, mass 73-90 kg) were tested, walking on a treadmill at 1.3 m/s. Metabolic rate (VO2, Physio-Dyne Max-II) was averaged over the last 3 minutes of each 7 minute walking trial to allow subjects to approach steady state. Ground reaction forces were also measured in 6 identical over-ground trials, and computed work performed on the body center of mass by each leg (see, e.g., Donelan, J M, et al. J Exp. Biol. 205: 3717-3727, 2002; herein incorporated by reference in its entirety). Push-off was defined as positive work by the trailing leg during double support, and collision as simultaneous negative work by the leading leg. Experimental conditions included normal walking, CESR prosthesis, and SACH prosthesis.

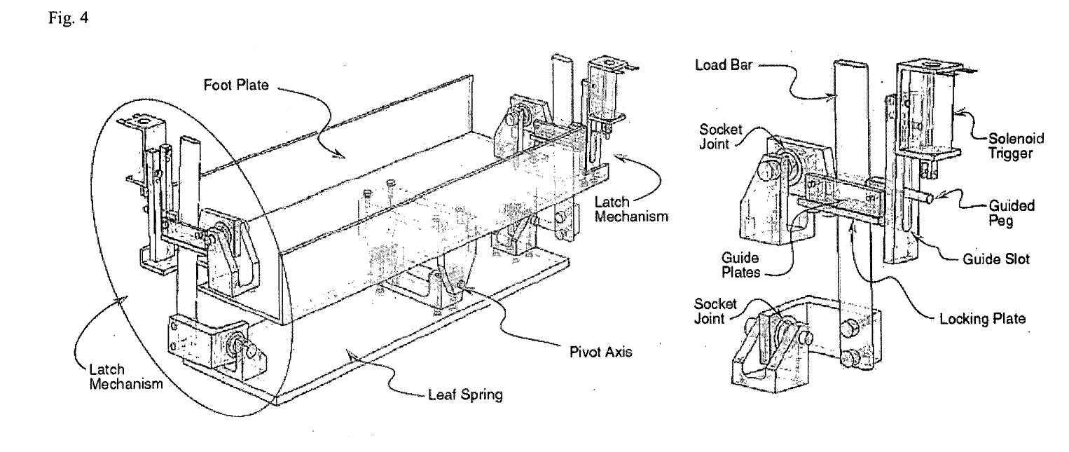

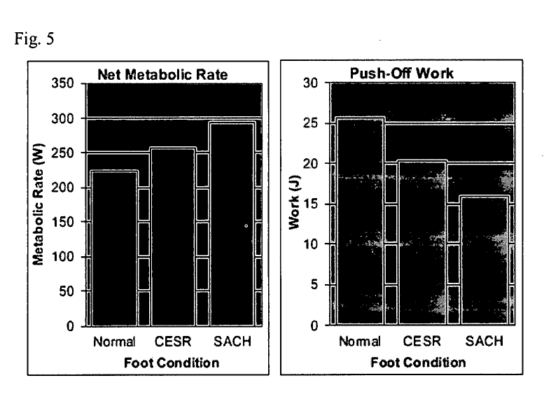

Walking with the SACH foot resulted in a 69 W increase in metabolic rate over normal walking, or about 31% (p<0.005, FIG. 5). This increase is consistent with results for amputees, though simulator mass and height may also have contributed to metabolic cost. With the CESR foot, subjects used 36 W less metabolic power than with the SACH foot (p<0.005). The CESR foot appears to partially compensate for the loss of push-off (FIG. 5). Work produced by the trailing leg with the CESR during push-off was 27% greater than that with the SACH foot (p<0.02). Both prostheses produced lower pushoff and greater collision or load acceptance forces than in normal walking (FIG. 6). The mechanical power capacity of the CESR foot was about 14 W, not all of which was successfully returned at push-off. Newer prototypes have been constructed that may improve on energy return.

A prototype prosthetic foot that stores and returns mechanical energy during successive step-to-step transitions was developed, significantly reducing metabolic energy consumption compared to a conventional prosthesis. Simultaneous positive and negative work during the step-to-step transition seems to be a significant determinant of the metabolic cost of walking, a determinant with clinical applications.

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A prosthetic device comprising
a toe plate and a heel plate, said toe plate and heel plate pivotably attached to one-another;
a spring disposed between said toe plate and said heel plate, wherein exertion of force on said toe or heel plate compresses said spring; and
at least one latch attached to said toe plate or said heel plate such that when said spring is compressed, said at least one latch engages said toe plate and/or said heel plate to maintain compression of said spring thereby storing energy that can be released upon disengagement of said latch.

2. The prosthetic device of claim 1, further comprising a microprocessor, said microprocessor configured to control said disengagement of said latch.

3. The prosthetic device of claim 1, wherein said prosthetic device is configured for attachment onto a leg.

4. The prosthetic device of claim 3, wherein said leg is an amputated leg.

5. The prosthetic device of claim 1, wherein said exertion of force onto said toe plate or said heel plate corresponds to a stepping down movement.

6. The prosthetic device of claim 1, wherein said microprocessor controlled latch disengagement is timed to match a lifting off motion during walking.

7. The prosthetic device of claim 1, wherein said microprocessor is a micro-electrical mechanical system.

8. The prosthetic device of claim 1, wherein said microprocessor is battery powered.

9. The prosthetic device of claim 1, wherein said microprocessor controlled latch disengagement pushes said toe plate in a plantarflexion direction.

10. The prosthetic device of claim 1, wherein said toe plate and heel plate is constructed of a carbon fiber and resin composite.

11. The prosthetic device of claim 1, wherein said prosthetic device is designed for placement within a shoe.

12. The prosthetic device of claim 1, wherein said microprocessor controlled latch disengagement permits the release of energy collected at said heel plate upon said toe plate.

13. A method of facilitating walking with a prosthetic foot comprising:
a) providing a prosthetic foot comprising a toe plate and a heel plate, a spring disposed between said toe plate and said heel plate, the compression and release of said spring controlled by a microprocessor;
b) allowing a force to be exerted on said heel plate such that said spring is compressed; and
c) via said microprocessor, releasing said spring such that said energy captured upon compression of said spring is released via said toe plate.
